(19) United States
(12) Patent Application Publication
(10) Pub. No.: US 2008/0294247 A1
(43) Pub. Date: Nov. 27, 2008

(54) Prosthetic Heart Valve

(75) Inventors: Jun Yang, Dove Canyon, CA (US); Van Huynh, Garden Grove, CA (US); Michael Estes, Lafayette, CA (US)

Correspondence Address: JACKSON & CO., LLP, 6114 LA SALLE AVENUE, #507, OAKLAND, CA, 94611-2802, US

(73) Assignee: MEDICAL ENTREPRENEURS II, INC., Scotts Valley, CA (US)

(21) Appl. No.: 11/754,250

(22) Filed: May 25, 2007

(51) Int. Cl. A61F 2/24

(52) U.S. Cl. 623/2.12

(57) ABSTRACT

The present invention includes prosthetic heart valves having flexible leaflets and methods for fabricating the valves which improve upon the prior art.

### FIELD OF THE INVENTION

The present invention is directed to prosthetic heart valves having flexible leaflets made of tissue or synthetic materials, and is also directed to improved methods of making such valves.

### BACKGROUND OF THE INVENTION

The human heart has four major valves which control the direction of blood flow in the circulation. The aortic and mitral valves are part of the “left” heart and control the flow of oxygen-rich blood from the lungs to the body, while the pulmonic and tricuspid valves are part of the “right” heart and control the flow of oxygen-depleted blood from the body to the lungs. The aortic and pulmonic valves lie between a pumping chamber (ventricle) and major artery, preventing blood from leaking back into the ventricle after it has been ejected into the circulation. The mitral and tricuspid valves lie between a receiving chamber (atrium) and a ventricle preventing blood from leaking back into the atrium during ejection.

Heart valves may exhibit abnormal anatomy and function as a result of congenital or acquired valve disease. Congenital valve abnormalities may be well-tolerated for many years only to develop into a life-threatening problem in an elderly patient, or may be so severe that emergency surgery is required within the first few hours of life. High blood pressure may also lead to cardiac valve abnormalities. Acquired valve diseases include degenerative processes (e.g., Barlow's Disease, fibroelastic deficiency), inflammatory processes (e.g., Rheumatic Heart Disease) and infectious processes (e.g., endocarditis). In addition, damage to the ventricle from prior heart attacks (i.e., myocardial infarction secondary to coronary artery disease) or other heart diseases (e.g., cardiomyopathy) can distort the valve's geometry causing it to dysfunction.

Since heart valves are passive structures that simply open and close in response to differential pressures on either side of the particular valve, the problems that can develop with valves can be classified into two categories: (1) stenosis, in which a valve does not open properly, and (2) insufficiency (also called regurgitation), in which a valve does not close properly. Valve stenosis is present when the valve does not open completely causing a relative obstruction to blood flow. Valve regurgitation is present when the valve does not close completely causing blood to leak back into the prior chamber. Stenosis and insufficiency may occur concomitantly in the same valve or in different valves. Both of these conditions increase the workload on the heart and are very serious conditions. The severity of this increased stress on the heart and the patient, and the heart's ability to adapt to it, determine whether the abnormal valve will have to be surgically replaced or, in some cases, repaired. If left untreated, these conditions can lead to debilitating symptoms including congestive heart failure, permanent heart damage and ultimately death.

Dysfunctional valves can either be repaired, with preservation of the patient's own valve, or replaced with some type of mechanical or biologic valve substitute. Since all valve prostheses have some disadvantages (e.g., need for lifelong treatment with blood thinners, risk of clot formation and limited durability), valve repair, when possible, is usually preferable to replacement of the valve. Many dysfunctional valves, however, are diseased beyond the point of repair.

Dysfunction of the left-sided valves—the aortic and mitral valves—is typically more serious since the left ventricle is the primary pumping chamber of the heart. The aortic valve is more prone to stenosis, which typically results from buildup of calcified material on the valve leaflets and usually requires aortic valve replacement. Regurgitant aortic valves can sometimes be repaired but are usually replaced. In modern societies, the most common mitral valve pathologies involve regurgitation due to gross billowing of leaflets to relatively minor chordal lengthening as well as ischemic disease. In the majority of these cases, the mitral valve leaflets are soft and pliable, and can be retained over the long-term in various repair procedures. However, in third world countries and in centers with high rates of immigration from third world countries, the most common pathology or condition is rheumatic mitral valve disease. This produced thickened, impliable leaflets with grossly deformed chords, or chordae tendinae, often combined with fusion of the two leaflets. Rheumatic valve are not suitable for any type of repair procedure and, accordingly, are almost always replaced.

Because the demands on the right side of the heart are significantly less than on the left, dysfunctions involving the pulmonic and tricuspid valves are far less common. The pulmonic valve has a structure and function similar to that of the aortic valve. Dysfunction of the pulmonic valve is nearly always associated with complex congenital heart defects. Pulmonic valve replacement is occasionally performed in adults with longstanding congenital heart disease. The anatomy and function of the tricuspid valve are similar to that of the mitral valve. It also has an annulus, chords and papillary muscles but has three leaflets (anterior, posterior and septal). The shape of the annulus is slightly different, more snail-shaped and slightly asymmetric.

Prosthetic heart valves can be used to replace any of the heart's valves. Two primary types of heart valve prostheses are known. One is a mechanical-type heart valve which uses a pivoting mechanical closure or a ball and cage design to provide unidirectional blood flow. The other is a “bioprosthetic” valve which is constructed with leaflets made of natural tissue and which function much like the leaflets of the natural human heart valve in that they imitate the natural action of the heart valve leaflets, e.g., they seal against each other or coapt between adjacent tissue junctions known as commissures. Another type of prosthetic valve has a structure similar to that of the bioprosthetic valves but whose leaflets are made from flexible synthetic material.

Each type of prosthetic valve has its own advantages and drawbacks. Presently, mechanical valves have the longest durability of available replacement heart valves. However, implantation of a mechanical valve requires a recipient to be prescribed anticoagulants to prevent formation of blood clots. Continuous use of anticoagulants can be dangerous, as it greatly increases the user's risk of serious hemorrhage. In addition, a mechanical valve can often be audible to the recipient and may fail without warning, which can result in serious consequences, even death.

In contrast, prosthetic valves having bioprosthetic and/or synthetic leaflets are flexible and silent, and those employing natural tissue leaflets do not require the use of blood thinners. However, naturally occurring processes within the human body may stiffen or calcify the leaflets over time, particularly at high-stress areas of the valve such as at the commissure junctions between the valve leaflets and at the peripheral leaflet attachment points or “cusps” at the outer edge of each leaflet. Further, the valves are subject to stresses from constant mechanical operation within the body. In particular, the leaflets are in tension when in a closed position and are in compression when in an open position. Accordingly, these types of prosthetic valves wear out over time and need to be replaced. Bioprosthetic and synthetic leaflet heart valves are also considerably more difficult and time consuming to manufacture than mechanical heart valves as they are made substantially by hand by highly trained and skilled personnel.

Bioprosthetic valves include homograft valves which include wholly harvested valves from human donors or cadavers; allograft valves which include biomaterial supplied from human cadavers; autologous valves which include biomaterial supplied from the individual receiving the valve; and xenograft valves which include biomaterial obtained from non-human biological sources including pigs, cows or other animals.

Currently available xenograft valves are constructed either by sewing the leaflets of pig aortic valves to a wire frame/form or stent (to hold the leaflets in proper position), or by constructing valve leaflets from the pericardial sac (which surrounds the heart) of cows, horses, pigs or other animals, and sewing them to a wire frame/form which in turns is coupled to a support stent or ring, often referred to as a pericardial valve. An example of a commercial valve having the latter configuration is the Carpentier-Edwards Perimount™ Pericardial Valve. That valve's stent has an upper surface “matching” the lower surface of the wireform between which the edges of the leaflets are sandwiched. In either of these types of xenograft valve embodiments, the wire frame/stent is constructed to provide a dimensionally stable support structure for the valve leaflets which imparts a certain degree of controlled flexibility to reduce stress on the leaflet tissue during valve opening and closure. The wire frames/stents are covered with a biocompatible cloth (usually a polyester material such as Dacron™ or PTFE.) which provides sewing attachment points for the leaflet commissures and cusps. Alternatively, a cloth covered suture ring can be attached to the wire frame or stent to provide an attachment site for sewing the valve structure in position within the patient's heart during a surgical valve replacement procedure. A number of prosthetic tissue valves have these constructs are described in U.S. Pat. Nos. 4,106,129, 4,501,030 4,647,283, 4,648,881, 4,885,005, 5,002,566, 5,928,281, 6,102,944, 6,214,054, 6,547,827, 6,585,766, 6,936,067, 6,945,997, 7,097,659 and 7,189,259 and U.S. Published Patent Application Nos. 2003/0226208 and 2006/0009842, which are herein incorporated by reference in their entireties.

While iterative improvements have been made over the last couple of decades, existing tissue valves are not without their shortcomings. One such shortcoming is the mismatch in size and mass between opposing surfaces of the wireform and stent. The mismatch is often due to the variabilities in the shape of the stent ring. Prior art stents are fabricated from a length of material which is formed or bent into circular configuration and whose ends are welded together. The forming and welding processes make the stent susceptible to “spring-back”, i.e., slight deformation undergone by the ring into a less than circular shape overtime. The tension applied to the stent upon suturing it together with the wireform, and that experienced during normal functioning of the valve, makes the stent further susceptible to spring-back. As illustrated in FIG. 1, the mismatch 2 exists between the circular wireform 4 and the less-than-circular stent ring 6. This mismatch 2 often leads to the wireform 4 becoming offset in either direction from the stent ring 6, which in turn leads to instability between the components. The instability results in uneven stress points, particularly on the valve leaflets, and subsequent expedited wearing of the valve.

Another shortcoming of the construct of existing bioprosthetic tissue valves is the potential for clot formation within the confines of the covering placed over the wireform and stent ring. This is best explained with reference to FIG. 2 which illustrates a cross-sectional side view of a prior art bioprosthetic valve at a commissure point (the wireform is not illustrated) when the commissure is subject to the natural forces exerted by the leaflets when in a closed position. To reinforce the wireform-stent assembly, commissure extensions or support members 8 are often incorporated into the valve at each of its commissures. The support members 8 are elongated protrusions which extend upward (towards the outflow opening of the valve) from the stent ring 6 and reside substantially within the confines of the space formed between the stent ring 6 and the wireform (not shown) at the valve's commissure points. These commissure pieces are commonly made of material that is relatively stiff but flexible (bendable), e.g., acetate material sold under the trade name MYLAR. As such, the pieces are able to flex, bend or deflect slightly inward upon the application of the radially inward force exerted on the valve leaflets and the resulting tension placed on the valve commissures under natural operating conditions, e.g., blood backflow pressure. When this deflection occurs, a pocket 7 may be formed between the cloth covering 5 and the commissure supports 8 in which thrombus may form and impede blood flow and valve function.

Accordingly, there is still room for improving the performance and stability of tissue heart valves and for improving the techniques for fabricating the valves. The present invention seeks to address the aforementioned shortcomings while maintaining desirable structural and functional features and ensuring functional longevity of the valve.

### SUMMARY OF THE INVENTION

The present invention includes prosthetic heart valves and methods for fabricating them. The subject prosthetic heart valves include a stent structure, a wireform and flexible valve leaflets. The stent structure includes a ring-like base and commissure extensions extending from the base in the valve's outflow direction. The wireform is operatively coupled to the stent structure at its outflow end. The leaflets are formed from flexible biocompatible materials, including biological tissue, such a pericardial tissue, and/or synthetic material, such as polyurethane, or a combination thereof.

The subject valves incorporate various improvements to address and overcome the shortcomings of prior art tissue valves. Certain of these improvements address the problem of “mismatching” that can occur between the wireform and the stent. For example, in one variation of, the stent's thickness dimension (i.e., the dimension between the stent's outer diameter and inner diameter) is made to be equal to or greater than the wireform's diameter dimension. In other variations the stent structure has an outflow surface having a dimension greater than the dimension of its inflow surface. In certain embodiments, the ratio of the stent's outflow surface dimension to the stent's inflow surface dimension may be 1:1 to at least about 8:5 or greater. In another variation, the stent's outflow surface is provided with depressions within the cusp portions to accommodate the diameter dimension of the wireform. Still yet, in other variations, the stent is formed in a manner such that its structure is seamless and has a diametrical shape that remains substantially constant under normal functioning of the valve. Further, the valve's wireform may have a diametrical shape substantially the same as that of the stent structure such that the wireform and stent structures are spaced apart a constant distance from each other, and whereby that spacing remains constant under normal functioning of the valve.

Certain other improvements provided by the present invention address the problem of thrombus formation within the confines of the covering which is placed over the valve's wireform and stent. In particular, the subject improvements minimize or prevent, among other things, the formation of a pocket between the covering and the inner surface of the stent's commissure extensions when the extensions are tensioned inward by the forces imposed on the valve under normal operating conditions.

In one variation of the inventive prosthetic valves, the stent structure has commissure extensions aligned within the commissures peaks of the wireform wherein the extensions are angled slightly inward to define a pre-fixed angle, typically within the range from about 0° to about 10°, with an inner wall of the stent. In this way, the range of motion which the commissure extensions are subject to is minimized, thereby minimizing the likelihood of the formation of a pocket between the covering and the stent wall. Angling of the commissures extensions may be accomplished by coupling separately formed commissure extensions to the stent base by mechanical means, such as a stitch, wherein their coupling defines a flexible joint. Alternatively, the extensions may be monolithically formed with the stent at the prefixed or predefined angle. In either case, the flexible point of joinder between the stent commissures and the stent base allow the commissures to flex or bend inward when subject to the normal operating forces exerted on the valve and its leaflets. To further ensure against the formation of a pocket between the cloth material and the inner surface of the commissure extensions, covering is provided substantially flush with the inner surface. This may be accomplished by the placement of a stitch between the two.

The methods of the present invention include fabricating a prosthetic valve where the stent structure, at least in part, is molded to have a shape that substantially matches that of the wireform. Such methods may further include molding the commissure extensions from the same mold as the stent base to form a monolithic structure. Other valve fabrication methods of the present invention include forming or providing the stent's commissure extensions at an angle to the inner wall of the stent. In other embodiments, the commissure extensions are separately formed from the stent's base and then coupled thereto in a manner to provide a flexible joint between each commissure extension and the stent base.

Other features, objects and advantages of the present invention will become more apparent from the following description taken in conjunction with the accompanying drawings.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Also for purposes of clarity, certain features of the invention may not be depicted in some of the drawings. Included in the drawings are the following figures:

FIG. 1 is a schematic illustration of a top view of a prior art bioprosthetic valve where the solid line represents a wireform and the dashed line represents a stent structure;

FIG. 2 is a cross-sectional side view of a commissure extension of a prior art prosthetic valve at a commissure point;

FIG. 3A is a side view of an assembled wireform and stent structure of a prosthetic valve of the present invention;

FIG. 3B is a cross-sectional view of the valve assembly of FIG. 3A taken along the lines B-B of FIG. 3A;

FIG. 3C is an enlarged end view of the cross-section of the valve assembly defined by circle C of FIG. 3B;

FIG. 3D is an enlarged cross-sectional view of the valve assembly taken along the lines D-D of FIG. 3B; and

FIG. 4 is a cross-sectional side view of the base of the cusp portion of a valve assembly of the present invention

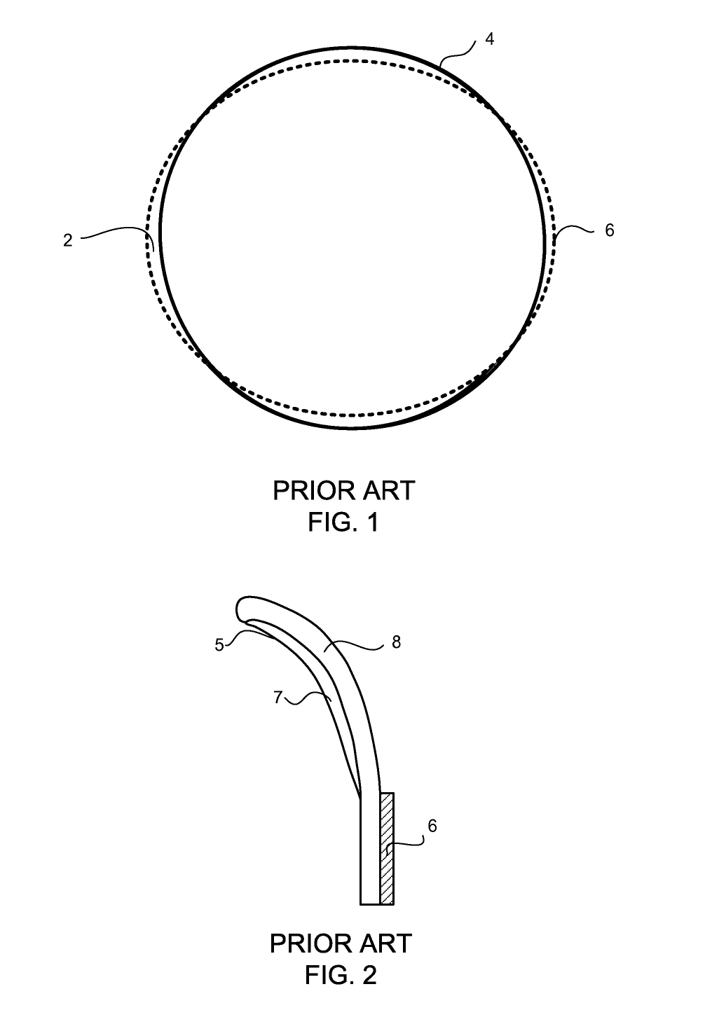

### DETAILED DESCRIPTION OF THE INVENTION

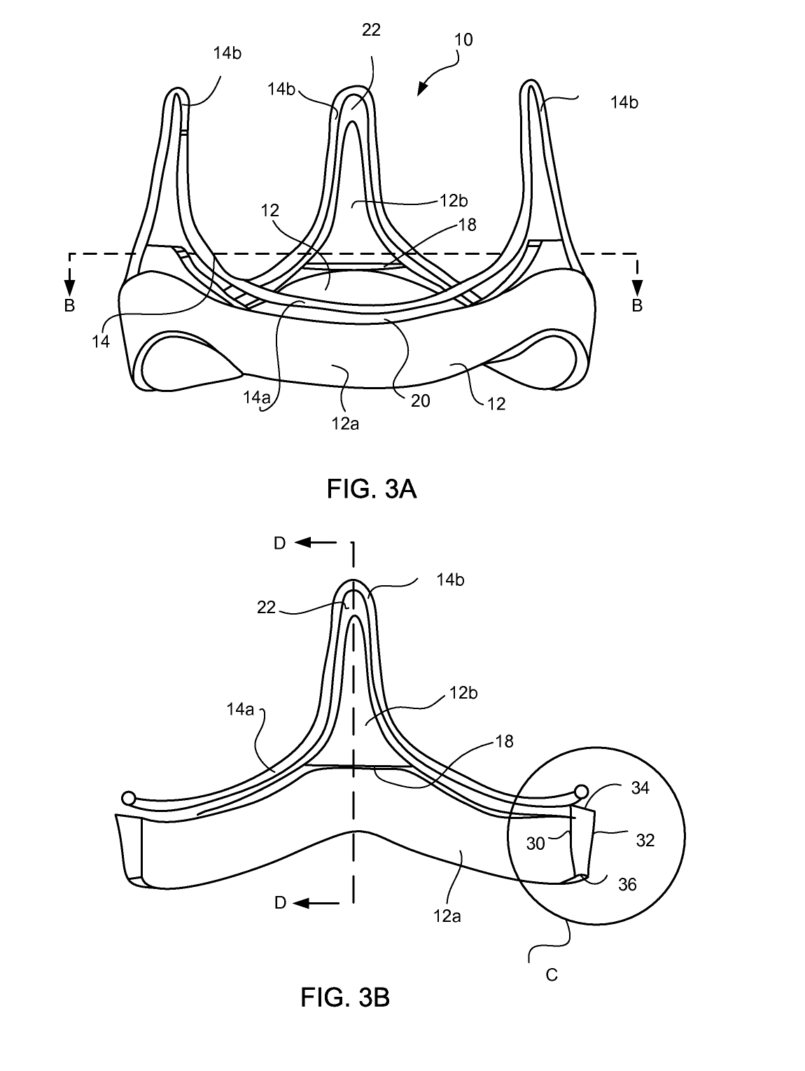

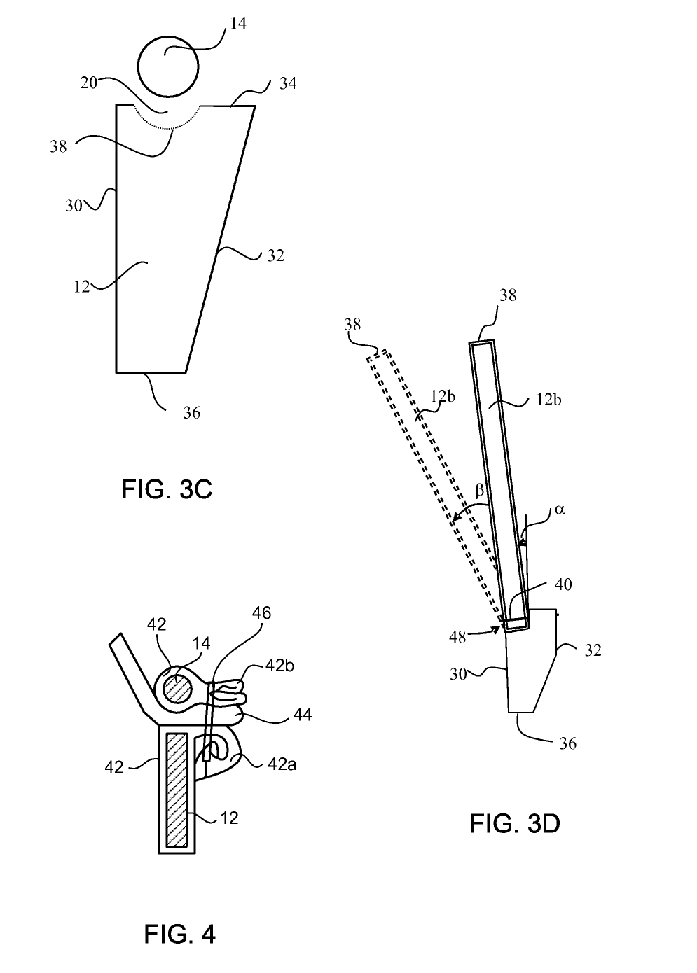

The present invention will now be described in greater detail with reference to FIGS. 3A-3D and 4, and by way of the following description of exemplary embodiments and variations of the novel devices, systems and methods. The invention generally includes an implantable prosthetic heart valve 10 having an annular stent in the form of a ring 12 and an annular wireform or frame 14 wherein the stent and wireform have substantially similar diameters. The wireform has an alternating pattern of arcuate cusps 14a and upstanding commissures 14b, whereby the number of each is typically three so as to most closely match the structure and function of the natural heart valve, e.g., the aortic valve, which it is intended to replace (although a three-leaflet valve of the present invention is also suitable to replace bicuspid valves, e.g. mitral valves). This undulating pattern mimics the natural contour of leaflet attachment and serves to support the prosthetic leaflets (not shown) within the valve. Stent 12 has a closely-matched pattern of cusp portions 12a and commissures 12b which are aligned with the corresponding cusps 14a and commissures 14b of wireform 14 (with a small portion 22 of the tip of the commissure left open or unoccupied) when the stent and wireform are operatively coupled together. The end of valve 10 having the commissure components 12b, 14b defines the outflow end of the valve, with the opposite end being the inflow end.

As with many conventional prosthetic tissue valves, prior to operative coupling of the wireform 14 to the stent 12, a tissue leaflet subassembly (not shown) is first applied, mounted and secured to the wireform 14 which has been covered with a cloth material 42 (see FIG. 4). The combined tissue-wireform structure is then secured to stent structure 12, with the leaflet tissue edges 44 sandwiched therebetween, to form the assembled valve 10. As illustrated in FIG. 4, ring 12 is also separately covered with cloth material 42. A portion of the cloth material, with both the stent and the wireform extends radially outward from the components to form tabs 42a and 42b, respectively, which provide a means for suturing 46 the two components together. When operatively coupled together, wireform 14 resides above stent 12 whereby the wireform is aligned with and tracks over the top or outflow surface 34 of stent 12. In a fully assembled valve having an integrated leaflet subassembly, the gap or spacing 20 defined between the two components is occupied by the tissue edges 44 of the leaflets over the entire length of the gap 20. As mentioned previously, the valve may be configured to be directly secured to the natural valve annulus or may otherwise be attached to a suture ring (not shown) which is attached to the natural valve annulus.

The various techniques and materials used to form the leaflets, form/bend wireform 14, fabricate the stent 12, and mount and couple the various components together, many of which are described in the patent documents incorporated by reference above, are well known and understood by those skilled in the art. For example, the tissue leaflets may be cut from harvested tissue, such as bovine pericardium. The cloth material used to cover the wireform and stent may be DACRON™ or another suitable textile material. Wireform 14 may be made of a cobalt nickel alloy wire (made by Elgiloy Ltd Partnership) commonly used for such wireforms, and stent 12 may be fabricated from a machined metal or a machined or molded plastic material (e.g., DELRIN™).

An advantage of the present invention in employing a molded stent ring, is that the stent structure is seamless (unlike the joint that is unavoidably formed when welding the stent) and the shape of the stent can be more accurately formed into the desired shape, and thus, be more accurately matched with that of the wireform. As such, with the respective shapes very closely matched, the bulkier, heavier stent component does not deform the weaker, lighter wireform when the valve is subject to the forces exerted on it during the valve fabrication process, e.g., when the stent and wireform are sutured together. For example, without such a close matching, when a stitch is applied between the cloth coverings of the two components is too tight or not tight enough, there is a tendency for the wireform to become uncentered with respect to the outflow end surface 34 of ring 12 (see FIG. 4 illustrating the wireform evenly centered with respect to the outflow surface of the ring). This component matching also ensures that the wireform and stent remain uniformly spaced from each other, i.e., the spacing between them is constant about the entire valve, thereby evenly distributing the compression force on the tissue positioned therewithin. Evenly-distributed forces on the valve as a whole and on the leaflet tissue in particular are intended to minimize the risk of premature wearing of the valve.

Another feature of the invention which, either alone or in conjunction with the closely matched shapes of the stent and wireform components, assists in maintaining the proper alignment and centering of the wireform with respect to the outflow end surface of the stent is the relative thickness of the stent's outflow surface 34 to the diameter of the wireform. Typically, conventional bioprosthetic valves have a wireform diameter of about 0.020″ to about 0.030″ while the thickness of the stent is about 0.015″. With these relative sizes, the wireform, when subject to both the natural forces exerted on the valve by blood flow as well as the tensions imposed by the stitching formed to secure the wireform to the stent, has a tendency to overhang the stent's outflow surface 34. This overhang may occur on either the inner stent surface 30 or the outer stent surface 32. To address this concern, the subject valve stents may have outflow stent surfaces 34 which have a thickness equal to or greater than that of the wireform diameter. For example, the outflow surface may have a thickness in the range from about 0.020″ to about 0.1″. As such, a “shoulder” is provided on the stent surface to accommodate any slippage or movement of the wireform thereon, making the centering of the wireform on the stent surface more easily accomplished and sustainable.

Another optional feature of the subject valve stents, as illustrated in FIG. 3C, is the provision of a depression or groove 38 within outflow surface 34 of the stent's cusp portions. The depressions may have any suitable cross-sectional profile, e.g., wedge shaped, rounded, etc., and have a dimension, e.g., radius of curvature, sufficient to accommodate that of the wire's diameter (or that of the radius of the wire with a cloth covering.

Those skilled in the art will appreciate the desire to maintain a valve opening which is as wide as possible without comprising the function and stability of the valve. As such and taking into consideration the supra-annular positioning of the valve of the present invention relative to the natural valve annulus, the subject valves provide thicker stent outflow surfaces while maintaining as wide a blood flow path through the valve. This is accomplished by selecting a stent cross-sectional shape which tapers from the outflow surface 34 to the inflow surface 36, i.e., the outflow surface 34 of the stent is greater than the corresponding inflow surface 36 of the stent. In the illustrated embodiment of FIG. 3C, the outflow surface 34 is thicker or greater than the inflow surface 36. In certain variations, the outflow to inflow surface ratio may be from about 1:1 to at least about 8:5, but may be greater or smaller depending on the application. In one embodiment, the outflow surface has a dimension or thickness of about 0.040″ and the inflow surface has a dimension or thickness of about 0.025”.

In addition to the relative sizing of the outflow and inflow stent surface, the particular shape of the stent's cross-section may be designed to enhance flow dynamics. For example, the cross-sectional shape of the stent ring of FIG. 3C is somewhat trapezoidal with inner surface 30 being substantially parallel to the direction of flow while outer surface 32 is angled outward to define a ledge or shoulder that extends toward the natural valve annulus. In this way, the added thickness of the outflow surface 34 is accommodated without reducing the effective orifice area. While the illustrated embodiment shows an angled outer surface 32, a straight surface with other accommodating geometries integrated into the valve structure may be employed.

Stent 12 has commissure support members or extensions or posts 12b, each extending from a point of joinder with a stent base and which, when ring 12 is operatively coupled with wireform 14, are aligned within respective commissure portions of the wireform. Instead of being flush and lying within the same plane as base portion 12a of the stent as in existing bioprosthetic valve configurations, the commissure extensions 12b are angled slightly inward to define a fixed or predefined angle α, typically in the range from about 0° to about 10°, with inner stent wall 30 (see FIG. 3D). The flexibility of the material forming the support members 12b as well as the manner in which the support members are interfaced with the base portion of ring 12 enable the support members to give or bend within a limited range of motion, defined as angle β, when the extension members 12b are subject to the natural forces placed on the valve leaflets. Angle β is generally in the range from about 0° to about 45°, and is more commonly in the range from about 20 to about 5°. In this way, the stress placed on the support members 12b is minimized. In one variation, the commissure extensions 12b may be separately formed pieces which are respectively coupled to base 12a at designated commissure locations. The extensions may be coupled to the stent by stitching or other suitable means to define a flexible joint 48. Alternatively, the entire stent may be monolithically molded with the predefined angled α between the base 12a and extensions 12b, and provided with a living hinge to allow for bending within angle range β when subject to the tensions undergone by the leaflets. Optionally, to prevent any thrombus formation that may occur between the inner surface 38 of extension 12b and the cloth covering 38 upon inward flexing of the extension, a stitch 40 may be applied through or about the two to maintain the cloth covering substantially flush with inner surface 38. Alternatively, the cloth may be adhered or secured to the inner surface of the extension by any other appropriate means, such as by sonic welding.

Methods associated with the subject valve devices are also contemplated within the scope of the invention. The subject methods may include fabrication and/or assembly steps or activities, including but not limited to molding and/or machining of the stent ring, bending of the wireform, attachment of tissue to the wireform to form the valve's leaflets, suturing together of the wireform and stent, etc. Other methods provide steps and activities associated with or implicit to the use and implantation of the valves within the body.

Yet another aspect of the invention includes kits having at least one valve of the present invention. A kit may include various other components for preparing, delivering, implanting and securing the valve. The subject kits may also include written instructions for implantation of the devices. Such instructions may be printed on a substrate, such as paper or plastic, etc. As such, the instructions may be present in the kits as a package insert, in the labeling of the container of the kit or components thereof, or provided as an electronic data file stored on a suitable computer readable storage medium, e.g., CD-ROM, USB, etc.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

It must be noted that as used herein and in the appended claims, the singular forms “a”, “an”, and “the” include plural referents unless the context clearly dictates otherwise. Thus, for example, reference to “a string” may include a plurality of such strings and reference to “the tubular member” includes reference to one or more tubular members and equivalents thereof known to those skilled in the art, and so forth.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

What is claimed is:

1. A prosthetic heart valve comprising:
a wireform defining cusps and commissures;
a stent structure having a base and a plurality of commissure extensions aligned within the commissures of the wireform, wherein the commissure extensions are angled slightly inward to define an angle with an inner wall of the stent base.

2. The prosthetic heart valve of claim 1, wherein the angle is in the range from about 0° to about 10°.

3. The prosthetic heart valve of claim 1, wherein the intersection between each of the commissure extensions and the stent base defines a flexible point of joinder having a range of motion between about 0° and about 45°.

4. The prosthetic heart valve of claim 3, wherein the range of motion is between about 2 and about 5°.

5. The prosthetic heart valve of claim 4, wherein the commissure extensions are separately formed pieces wherein their respective points of joinder to the stent base are formed by mechanical means.

6. The prosthetic heart valve of claim 5, wherein the mechanical means comprises a stitch.

7. The prosthetic heart valve of claim 3, the commissure extensions are monolithically formed with the stent base and each point of joinder is defined by a living hinge.

8. The prosthetic heart valve of claim 1, further comprising:
a cloth material covering at least an inner surface of each commissure extension wherein the cloth material is substantially flush with the inner surface of the commissure extensions.

9. The prosthetic heart valve of claim 8, wherein a stitch maintains the cloth covering substantially flush with the inner surface of each commissure extension.

10. The prosthetic heart valve of claim 1, wherein the commissure extensions comprise Mylar.

10. A method for fabricating the prosthetic valve of claim 1, the method comprising providing the commissure extensions at a prefixed angle to the inner wall of the stent base.

11. The method of claim 10, wherein the step of forming comprises molding a monolithic structure comprising the stent base and the commissure extensions.

12. The method of claim 10, wherein the step of forming comprises coupling the commissure extensions to the stent base.
